(19) United States
(12) Patent Application Publication
(10) Pub. No.: US 2009/0121156 A1
(43) Pub. Date: May 14, 2009

(54) Apparatus and Method for Surface Preparation Using Energetic and Reactive Cluster Beams

(76) Inventors: John F. Mahoney, South Pasadena, CA (US); Julius Perel, Altadena, CA (US); James K. Finster, Pasadena, CA (US)

Correspondence Address: CHRISTIE, PARKER & HALE, LLP, PO BOX 7068, PASADENA, CA, 91109-7068, US

(21) Appl. No.: 11/884,517

(22) PCT Filed: Feb. 15, 2006
(86) PCT No.: PCT/US06/05678

§ 371 Date: Aug. 15, 2007

Related U.S. Application Data
(60) Provisional application No. 60/652,606, filed on Feb. 15, 2005.
(60) Provisional application No. 60/716,043, filed on Sep. 9, 2005.
(60) Provisional application No. 60/718,259, filed on Sep. 16, 2005.

(51) Int. Cl. A61N 5/00

(52) U.S. Cl. 250/492.2

(57) ABSTRACT

A method and apparatus for cleaning contaminated surfaces, especially semiconductor wafers, using energetic cluster beams is disclosed. In this system, charged beams consisting of microdroplets or clusters having a prescribed composition, velocity, energy and size are directed onto a target substrate dislodging contaminant material. The charged, high energy cluster beams are formed by electrostatically atomizing a conductive fluid fed pneumatically to the tip of one or more capillary-like-emitters.

### CROSS-REFERENCE TO RELATED APPLICATIONS

This application claims priority to and the benefit of U.S. Provisional Application Nos. 60/652, 606, filed Feb. 15, 2005; 60/716,043, filed Sep. 9, 2005; and 60/718,259, filed Sep. 16, 2005, the disclosures of which are incorporated fully herein by reference.

### BACKGROUND OF THE INVENTION

1. Field of the Invention

The present invention relates generally to modifying the surface of a target substrate or workpiece, and more particularly to apparatus and methods for cleaning, drying, texturing, and coating microtechnical substrates such as semiconductor wafers, micropackages, disk media, disk heads, and medical devices, using a microcluster beam that has been generated electrostatically, optionally neutralized, and directed toward the substrate surface, in order that the microclusters can dislodge and remove particles and films, deposit coatings, removing moisture, or texture.

2. Description of Prior Art

Cleaning, drying, coating, and texturing are surface preparation processes that are required for proper manufacturing in many microtechnical markets. For many years, substrates and workpieces have been combined into “batches”, and then processed by placing these batches into various chemical baths and rinse baths. As the effects of cross contamination and other factors have become more problematic, substrates and workpieces are now often processed as single units. Chemical and water sprays can be used in place of the immersion of substrates into liquid baths. Plasma processing is been used in some applications instead of wet chemicals.

Removal of thin films of water after rinse has been accomplished with a number of drying techniques. Air knives, substrate heating, and surface tension gradient (Marangoni) methods are typical.

As feature sizes become smaller, prior surface preparation equipment and methods become less effective. In the case of contaminant removal, end product yield is negatively affected, causing increased manufacturing costs. Current methods often involve large volumes of water and chemistries, some of which are hazardous to health and the environment. Disposal of hazardous waste can add significant costs to manufacturing.

During drying, small contaminants that may be trapped in thin films of water prior to evaporation, and can cause problems when deposited onto the substrate.

While prior methods may be effective in certain situations, there is a need for improved surface preparation apparatus and methods with the capability to deliver both kinetic and reactive processes to a surface. In addition, as the dimensions of features continue to decrease, a method for creating microdroplets that can react, release, lift, encapsulate, and evacuate debris of smaller sizes is needed.

The object of this invention is to allow producers of technical products, for example semiconductors, display panels, disk media, and medical devices, to be able to use a new, flexible set of equipment that will provide advances in surface preparation. Such advances include removal of smaller contaminants, improved workpiece flow through manufacturing, reduction of chemical usage and waste creation, and tighter integration with adjacent processes.

### SUMMARY OF THE INVENTION

The present invention provides apparatus and methods for surface preparation on substrates and workpieces. Surface preparation is performed by the interaction of a beam of microclusters that impinge upon the substrate or workpiece surface in order to clean, dry, coat, or texture the surface. A liquid solution is pre-mixed or mixed at point of use, then presented to a Fluid Control System that includes a fluid reservoir, optional point of use filtration, an electro-pneumatic fluid flow controller, and fluid distribution components. An electrohydrodynamic (EHD) Emitter Source Module aerosolizes the solution into microclusters using electrostatic forces. Optional beam conditioning electrodes may be included to direct or manipulate the microcluster beam. Once generated, the microcluster beam travels through the Transport Media, either vacuum, air, or gas, and goes through changes such as microcluster acceleration, breakup, or discharge. The microcluster beam impinges upon the substrate or workpiece Target Surface and performs the desired surface preparation though physical and/or chemical interactions. A Neutralizer may operate on the Target Surface and/or the microcluster beam in order to eliminate or reduce charging of the Target Surface. An Automation System composed of computer-based electronics, sensors, actuators, software, user interface, and inter-system communication monitors and controls the surface preparation process.

The electrohydrodynamic (EHD) process generates charged liquid clusters (droplets) from a liquid pool. The clusters are accelerated from the pool by the electric field that forms cone shaped emission sites.

Emission and particular mode depend upon the balance of several parameters that sustain the liquid shape at the tip of the nozzle during the process. The parameters include the spray solution characteristics that connect the solution to the applied electric field, such as conductivity, and those that relate to flow rate and affect the shape of the exposed solution. Other parameters involve the dimensions and shapes of the nozzle tips and the emitter electrodes. The two primary variables that allow for process control of a given emitter and solution are the solution flow rate (in the range of 0.1 to 0.8 μL/min) and the applied voltage (in the range from 3 to 15 KV) controlling the electric field.

The electric field at the emitter tip is controlled by applying voltage from a high voltage power supply to the solution stored in the reservoir container. The solution, being conductive, retains the applied voltage even when emerging at the nozzle tip, where the electric field forms the liquid shape. The spray mode is determined by the liquid shape, which in turn is formed by a balance between the liquid flow in addition to the electric field. The flow rate is controlled by gas pressure applied to the solution to drive it through tubing to the emitter.

There are several modes of operation including “burping”, which is unstable where it introduces mass at a rate that exceeds the cluster removal ability afforded by the electric field. The Taylor Cone grows to a level where it bursts and burps out a large amount of liquid. The cone re-forms in smaller dimensions and starts to again grow to repeat this periodic process. This mode is unstable and is not expected to be an efficient surface preparation mode.

The “single Taylor Cone” mode forms at a higher voltage (electric field) or conversely at a lower flow rate where the two balance such that the removal of clusters matches the mass delivered by the flow rate. In this mode a single spray site occurs at the end of the stable well-formed liquid cone. Photo 1 shows the single Taylor Cone mode of operation.

Multi-beam emission occurs when the voltage is increase, or conversely if the mass flow is decreased, a second cone is first formed, generally symmetrically spaced and on to multiple sites as the voltage is increased. The formation of more multiple emission sites is accompanied by a decrease of the liquid volume at the emitter tip. At somewhere close to five or six sites, they arrange around the edge of the emitter to form a “crown mode” of emission. This mode is generally stable over a wide range of voltages and flow rates. Photo 2 shows the crown mode of emission.

The cluster beam accelerated from the emission site by the electric field, carries away mass somewhat below the mass flow delivery rate because of evaporation of volatile solutions. It also carries away charge, producing a cluster beam current in the general range from 0.1 to 2.0 uA. The current is affected by both the voltage and especially the mass flow rate.

The beam shape is directly related to the emission mode. The single Taylor Cone mode forms a conical beam with angles from about 10 degrees to 90 degrees, with the angle increasing with the flow rate and voltage increases. The crown mode of emission produces separate beams generally symmetrically spaced in radial geometry. This is very evident in the crown mode, where anywhere from four to at least ten beams are symmetrically spaced ranging to over 100 degrees.

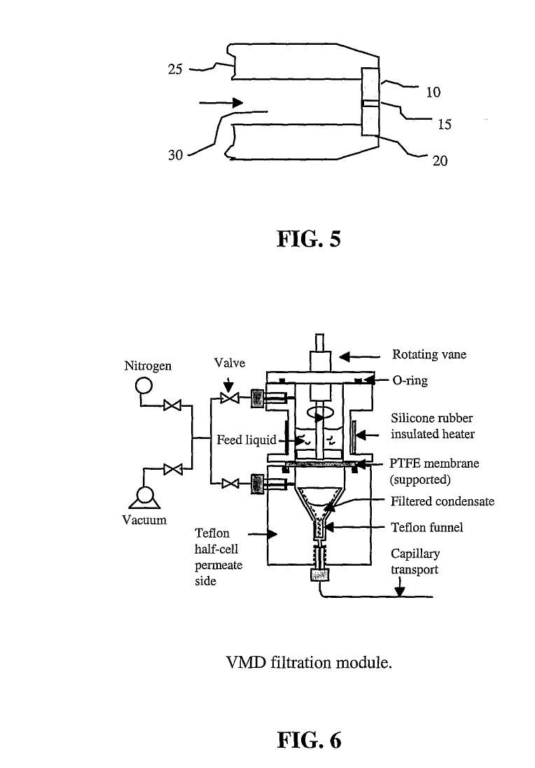

The above photos show the beam pattern of the nozzle of FIG. 5, for example. The orifice 15 is labeled in Photo 1 and the disc is labeled 11. The shape and mode of the beam pattern 10 are related to the charge placed on the liquid exiting orifice 15. In photo 1 the liquid exits orifice 15 and converges in liquid form to a throat 10a, After passing throat 10a, the liquid forms an aerosol 10b made up of electrically charged microdroplets. The kinetic (impact) energy of the beam 10 is the result of the charge on the microdroplets. If the target substrate is conductive, it can be grounded to prevent excessive charge buildup thereon.

### BRIEF DESCRIPTION OF THE DRAWINGS

The present invention, and various subcomponents within the invention, will be readily understood by the following brief descriptions in conjunction with the accompanying drawings.

### DETAILED DESCRIPTION OF THE INVENTION

The invention can be used in numerous applications.

One usage is the removal of contaminant particles from semiconductor wafers as in-process cleaning steps. Another usage is the removal of films, such as photoresist, anti-reflective coatings, or sacrificial layers, from semiconductor wafers as in-process cleaning steps. Another usage is the deposition of thin films onto semiconductor wafers through the accumulation of microclusters remaining on the substrate surface. Yet another usage is the removal of a final thin film of a liquid from a semiconductor wafer as a final drying step. Still another usage is texturing the surface of a semiconductor wafer to better prepare the surface for the adherence of a subsequent thin film deposition.

Additional usage is finishing and purging in various dryers used in drying wafers, panels, disk media, micropackages and other electronics substrates. In Marangoni and variants of such drying methods, microcluster beam purges using various trace solvents, surfactants, chemicals in high purity water become dry molecular-level clusters that sweep away trace residual, chemicals and moisture in a final purge.

In addition to semiconductor wafers, the same surface preparation processes may be performed on substrates or workpieces in other technical markets, such as satellite and aerospace components, sensors, crystal manufacturing for electronic systems, etc.

An additional usage is conversion of metals in liquid form, both molten and polymeric, for deposition on surfaces in uniform layers or via a focused emitter for spot deposition of interconnects and other forms of pads. One such application is metal fill of through-wafer vias in 3D packaging applications. Another application would be metalizing pads for ultrasonic, and related, bonding in modules, multi-chip modules, micropackages and disc/disk head assemblies.

An additional usage is conversion of liquid coating materials such as dielectrics, sealants, and faraday materials, to nano-clusters for deposition uniformly on surfaces and to seal sub-surface porosity. One such application is a nanometric layer of sealant on porous low-K dielectric to eliminate the absorption of various process materials and chemistries in subsequent steps. Another application would be coating discrete track recording disks with a final thin layer of diamond like carbon and subsequent perfluoropolyether lubricants.

An additional usage is a focused beam emitter that would provide an etchant beam to a point of contact with a laser beam for microslicing or scribing wafers, and other critically sensitive substrates, without the use of high-powered lasers and the exceptional heat and radiation produced.

An additional usage is a high-energy emission of microclusters to uniformly texture surfaces for further bonding of critical layers that might have a different thermal co-efficient that would impact layer bonding at performance temperatures. One such application is texturing semiconductor wafer substrates for epitaxial layers such as insulation layers that reduce power leakage. Another application is texturing the backside of wafers for thick dielectric bonding in 3D packaging where stacked packages would generate high-temperatures between wafer die. Spot texturing using a focused beam emitter could provide landing zone spot texturing on disk media as a clean-in-process texturing method that would eliminate significant post-cleaning prior to further processes.

An additional usage is removal of residuals and cleaning of modules, packages, and microassemblies that have complex surface dimensions. In various modules, components placed in surface mount have their contact pads underneath the package and cleaning must remove excess residuals, and their resident moisture, that form around pads that can cause bridges or shorts. One significant application is removal of non-lead bonding residuals often called HAIRS.

An additional usage applies to cleaning and preparing hard disk drive media substrates and related disks/discs for sputtering of various recording layers using various surfactants and/or solvents to remove hydrophobic and hydrophilic residuals and particles. This technique, done within vacuum chambers of the sputter equipment, eliminates significant washing, scrubbing, cleaning and drying prior to sputter done in traditional megasonic washers and brush scrubbers. At various steps in the sputter process there are additional usages in a) cleaning diamond like carbon layers for deposition of lubricant, b) cleaning final metal layers for sacrificial masking layers used in imaging discrete track recording (DTR) disk media, and c) creating clean, textured landing zones on the disk edge.

An additional usage is in processing head wafers, strips and heads, known as “sliders”, using the similar resist/strip, lift-off and related processes designed for use in semiconductor wafer and die processing. Removal of hydrophobic/hydrophilic residuals, resists, sacrificial layers, and adhesives are critical to cleaning the interface between the read/write recording head and the disk media during disk operations.

An additional usage is the delivery of new, low temperature chemicals in decontamination and sterilization, such as CIDEX by ASP-Johnson & Johnson, in removal of pathogen and pyrogen during production of catheters, stints, joint replacement, test vials and implantable electronics such as cardiac rhythm monitors.

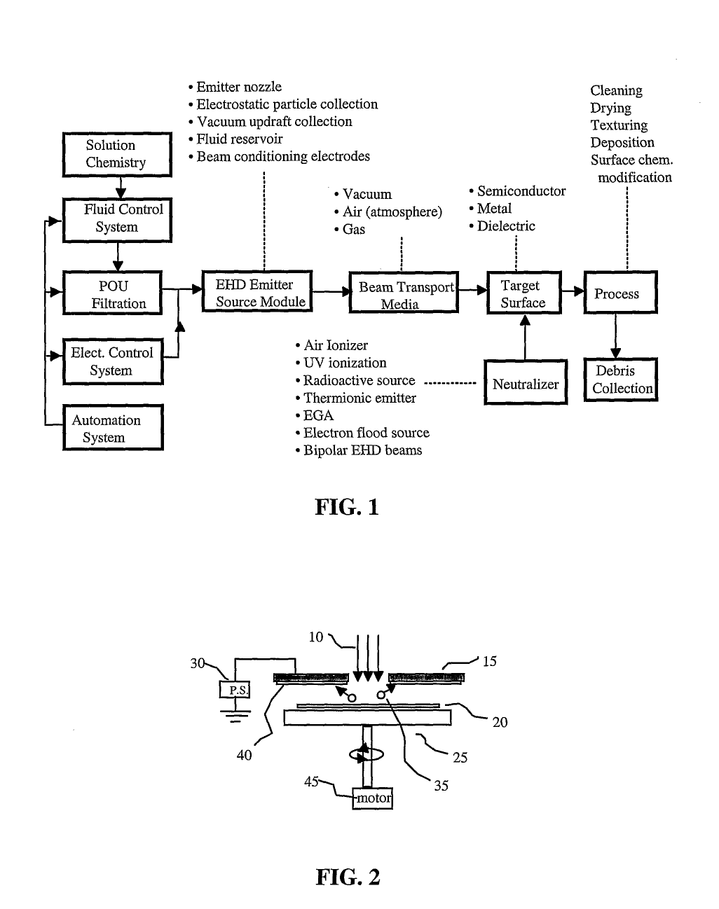

FIG. 1 shows the top-level block diagram of the invention. Subsequent figures and descriptions detail various configurations, aspects, and subsystems of the invention. In most, if not all of the configurations, the described emitter source module, beam transport media, and target surface are all enclosed in a housing isolated from the exterior environment. The housing protects the process from contaminants and permits operation in a vacuum or a specific gaseous environment.

### Electrostatic Collection

In the process of impacting a substrate with a charged microdroplet beam, collisions between the microdroplets and residual particles on substrates results in the removal of the residual particles. Removed particles will retain a charge, positive or negative, depending on the polarity of the charged microdroplet beam impacting the surface. By intentional charging of the impacted particles, electrostatic means can be used for efficient collection of the resuspended particles.

A substrate 20 (FIG. 2) containing contaminant particles is subjected to a beam 10 of charged microdroplets. After liftoff from the surface, the charged particles 35 are attracted to collection plates 15 by applying either an AC or DC voltage of opposite polarity to the sign of the particles by means of power supply 30. The collection plate may be coated with a dielectric film 40 consisting of paralyene or other suitable dielectric material to prevent particles from losing their charge and being re-attracted back to the substrate 20.

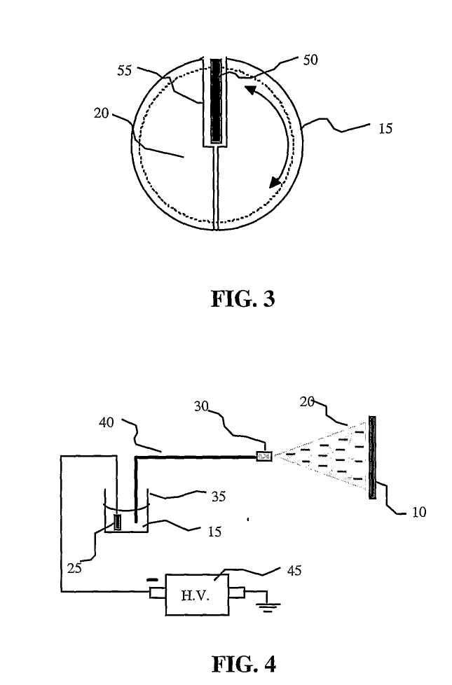

A slotted region 55 (FIG. 3) in the collection plates 15 allows for mounting a stationary linear slit or linear array of capillary nozzle emitters 50 for generating the charged microdroplet beam 10 which impacts the substrate. The substrate 20 is rotated by means of motor 45 under the collection plates 15.

### Negatively Charged Microdroplet Beam

Conventional electrohydrodynamic (EHD) and electrospray charged droplet emitters rely on the generation of positively charged microdroplet beams. Unless the target substrate is properly grounded, a means for supplying electrons is necessary to prevent substrate charging when exposed to a positive beam. Without neutralization, substrates will charge to high positive potentials. Therefore positively charged beams that impact insulating or semiconducting surfaces require a source of neutralization. However, by using a beam of negatively charged microdroplets, both insulating and grounded substrates will not significantly charge up. FIG. 4 illustrates a method for generating a beam of negatively charged microdroplets. A power source 45 is used to apply a negative voltage to an electrode 25 immersed in a reservoir 35 containing the electrolytic solution 15. The solution 15 which is electrostatically dispersed must contain a electrolyte or chemical species capable of placing negative charge on individual microdroplets. In the case of water or isopropyl alcohol, HCL is an example of an conductive additive able to supply the negative charge in the form of Cl⁻. Other solutions such as formamide are naturally conducting and may not require an additive to provide negative charge. When the solution is delivered to the emitter 30 by way of the transfer line 40, a beam of negative microdroplets 20 is formed that impact substrate 10.

Negative charges from the beam tend to charge an ungrounded, insulated substrate 10 negatively. On the other hand, secondary electrons emitted from the substrate after impact by the microdroplet beam 20 tend to charge the substrate positively. The interaction of the two charging mechanisms results in a charge balance that maintains the substrate at near zero potential. Consequently, the need for an electron neutralizer is eliminated which greatly simplifies surface preparation processes. However, if substrate 10 is electrically conductive, charge buildup thereon can be prevented by grounding substrate 10.

### Contamination-Free Emitter Design

When applying EHD microdroplet beams in the surface cleaning mode, it is paramount that the emitter structure (using linear slit or nozzle array emitters) does not add contaminants to the atomized solution. Otherwise, contaminants introduced by the emission process can be deposited on surfaces to be cleaned.

FIG. 5 shows a single emitter nozzle design for minimizing or eliminating contaminants introduced into the solution 30 during substrate cleaning. Features of the emitter design that minimize or prevent contaminants from entering the solution include small surface area to volume ratio of the support tube 25 compared to fused silica capillaries, non-flexibility of the support tube and low particle shedding from both the support tube and sapphire disc 10.

The emitter section of the nozzle is machined from a chemically inert sapphire (Al₂O₃) disc 10 containing a precision orifice 15. The orifice disc is sealed at its circumference 20 to a support tube 25. The support tube is of short length (≈3 to 4 inches) made of chemically inert and particle-free material preferably PEEK, Teflon or other non-conducting material exhibiting little or no particle shedding on contact with the EHD solution. The sapphire disc 10 is preferably 0.060 inches in diameter and 0.010 inches thick having an orifice about 10 micron in diameter. Existing nozzles made from metal or long lengths of fused silica have a tendency to shed particles—especially the latter which is frequently bent in handling and installation. The inner diameter of the support tube 25 is preferably about 0.030 inches in diameter.

Point of Use Filtration using Vacuum Membrane Distillation (VMD)

Liquid filtration is a critical requirement so that contaminants are not introduced as a by-product of the EHD atomization process. This concern is based on two factors: the infrastructure needed to acquire point-of-use semiconductor grade chemicals of sufficient purity (low particle levels below 0.2 μm) and inherent limitations on particulate retention efficiencies offered by flow-through membrane filters.

To circumvent these difficulties, a vacuum membrane distillation (VMD) process can be used to filter and purify liquids used in the electrohydrodynamic (EHD) cleaning process. VMD is a separation process that uses microporous hydrophobic membranes. The VMD filtration module design is shown in FIG. 6. The module is composed of two half-cells separated by a membrane. The upper half-cell (feed side) contains the liquid phase or feed solution. The lower half-cell (permeate side) is kept under vacuum at a pressure below the equilibrium vapor pressure of the liquid. After heating the unfiltered liquid on the feed side, the liquid vaporizes at one side of the membrane and the vapor diffuses across the pores of the hydrophopic membrane. Heating serves to increase the liquid vapor pressure providing the driving force. This driving force is further aided by applying a vacuum to the permeate side during the distillation cycle. On the permeate side of the distillation module, the vapor flux moves across the vacuum gap and is allowed to condense on a cold surface where the filtrate is recovered and delivered to the EHD cleaning head. VMD depends on the hydrophobic nature of the microporous membrane to prevent the liquid on the feed side from penetrating the membrane pores. Due to the absence of liquid transport, particles which are unable to evaporate cannot diffuse across the membrane pores. A Teflon rotating vane is placed in the feed side solution to stir the liquid for stimulating cross-flow across the membrane. This should prevent buildup of cake particles on the membrane that could reduce the permeate flux through the membrane pores. VMD offers rejection rates of macromolecules, colloids, submicrometer particles or other non-volatile constituents approaching 100%.

VMD has been used on a limited basis for the following: production of ultrapure water from salt solutions (desalination), removal of trace volatile organic compounds from waste water, extraction of dissolved gases, and concentration enrichment of non-volatile species on the liquid side of the membrane. The lack of general interest in VMD for particle filtration may, in part, arise from the requirement that solutions must not wet the hydrophobic microporous membrane. This limits VMD to processing water, aqueous solutions or other liquids that possess high surface tensions. Also, the mass flux or material throughput performance is not sufficiently high to render the process feasible for most industrial scale applications. For EHD cleaning applications, however, the quantity of liquid needed to be processed by VMD is extremely small and can take advantage of the limited throughput of a VMD apparatus. Calculations show that material transfer rates in a VMD apparatus can readily match or exceed the material consumed in the EHD cleaning process.

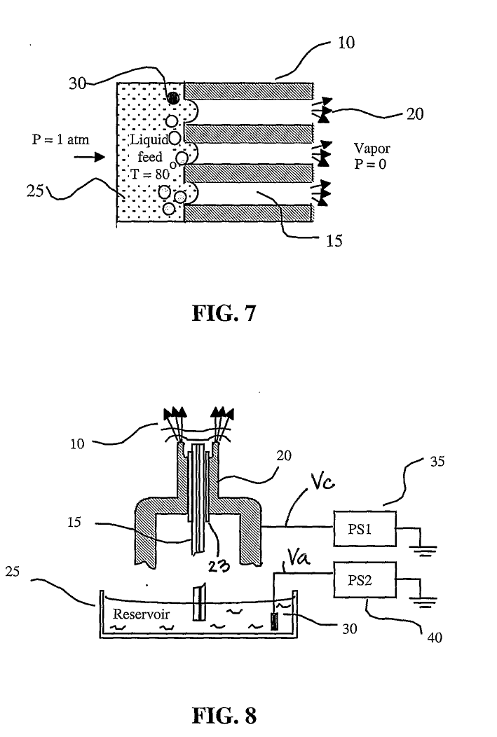

The most important criterion for the filtration process is that the liquid does not wet the membrane material; otherwise the pores would immediately fill with liquid and shutdown the filtration dynamics. Thus a non-wettable porous hydrophobic membrane 10 must be used as shown in FIG. 7. When operating, particles 30 cannot pass through the membrane pores 15 and remain in the upstream liquid 25. Only soultion vapor 20 passes through the membrane pores 15. Since wetting is favored when the membrane polymer has a high surface energy, a membrane must be selected with the lowest surface energy compatible with the VMD filtration process. Typically, for best operation, the membrane 10 should be about 150 microns thick having pore diameters about 0.2 micron. Table 1 lists the surface energies of several polymeric materials used in membrane construction. From this list, polytetrafluoroethylene (PTFE) has the lowest surface energy and would be the best choice of material.

TABLE 1
Surface energies of polymeric materials

| Polymer | Surface Energy (N/m) |
|---|---|
| polytetrafluoroethylene | 0.018 |
| polytrifluoroethylene | 0.024 |
| polyvinylidenefluoride | 0.030 |
| polyvinylchloride | 0.036 |
| polyethylene | 0.033 |
| polypropylene | 0.030 |
| polystyrene | 0.042 |

Since wettability is determined by the interaction between the liquid and the polymeric membrane material, a second important factor is the surface tension of the liquid. Wetting is favored when a liquid has a low surface tension. To avoid or minimize wetting of the polymeric membrane pores, any liquid used for electrospraying should have a high surface tension. Water, glycerol and formamide have high surface tensions compared to IPA, methanol and other alcohols. The surface tension of ethylene glycol has an intermediate value lying between water and the alcohol's (see Table 2). Closely connected to surface tension is the concept of wetting angle. To prevent pore penetration of the liquid, the contact angle between the liquid and the membrane surface should be >90 degrees.

TABLE 2
Liquid surface tensions.

| Liquid | Surface Tension (N/m) |
|---|---|
| Hydrogen Peroxide (35%) | 0.074 |
| Water | 0.073 |
| Glycerol | 0.063 |
| Formamide | 0.058 |
| Ethylene Glycol | 0.048 |
| IPA | 0.022 |
| Methanol | 0.022 |

### Emitter Microdroplet Size Control

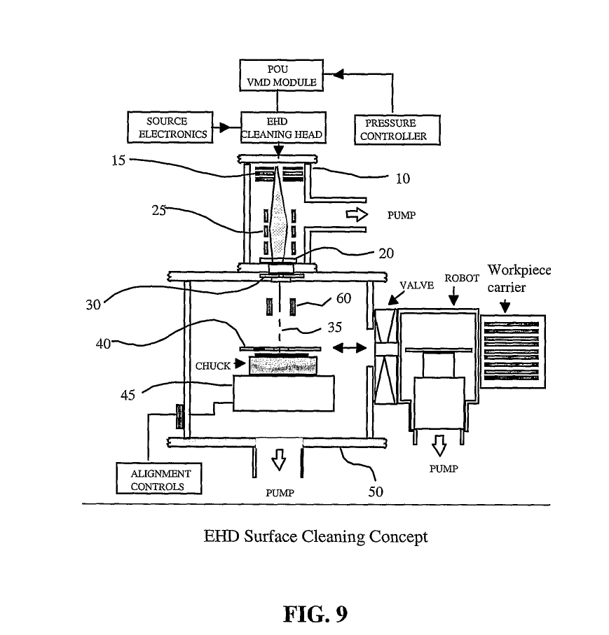

This inventive feature allows the microdroplet size to be varied without changing the impact energy of the microdroplets. In FIG. 8, the impact energy is controlled by the acceleration potential applied by means of power source 40 to a conductive electrode 30 immersed in a solution contained in reservoir 25. A second voltage is applied by means of power source 35 to a conducting emitter cap 20 that surrounds the microdroplet emitter 15. The surface of the solution in reservoir is exposed to the space within cap 20, which is sealed from the surrounding environment. A pump (not shown) pressurizes the space within cap 20 so charged droplets are formed at the exit of cap 20. The voltage applied to the conducting, tubular cap 20 that encircles the capillary emitter 15 is electrically isolated from the solution by an insulative sleeve 23. Whereas the applied solution voltage determines the final acceleration potential, Vₐ, the cap voltage 35 acts as a current control device by modifying the electric field 10 at the capillary tip. Varying the cap voltage, V_{C}, causes the emission current to increase or decrease while keeping the acceleration potential constant. When V_{C} is lowered below Vₐ, the emission current increases accompanied by a corresponding decrease in the average size of the emitted microdroplets. Raising V_{C} above Vₐ, results in a lower capillary emission current accompanied by a corresponding increase in the average size of the emitted microdroplets. Operation of the emitter in this configuration is analogous to the operation a vacuum tube triode. This unique arrangement for controlling the microdroplet beam current, hence the microdroplet size, at constant volumetric flowrate and acceleration potential is depicted in FIG. 8 (not to scale). Typically, the chemically inert solution electrode 30 consists of a large diameter gold or platinum wire. The capability of controlling the microdroplet emission current in real-time is an advantage for cleaning applications that require removal of both thin oxide or polymeric films and particles from substrates. Films are removed faster using high current (>1 μA), high charge-to-mass ratio microdroplet beams. Conversely, lower current beams, characterized by a larger-sized microdroplet distribution, are more effective for removing particles. The emitter assembly connected electrically as shown in FIG. 8 provides added flexibility for cleaning surfaces contaminated with more than one types of residue.

### Single-Emitter Sharpshooter Apparatus

A surface cleaning and preparation apparatus for detaching nanometer-size particles from photomasks, wafers and other critical surfaces is shown in FIG. 9. The basic method uses accurate X-Y coordinate mapping techniques that locate discrete particles that remain on surfaces before or after a primary cleans (wet, SCCO₂, e.g.)

By programming an X-Y stage, surface particles are positioned directly beneath a collimated EHD microdroplet beam and removed. Several benefits accrue from this cleaning concept and include:
1. The cleaning system uses a single EHD emitter reducing the size of the pumps necessary to evacuate the EHD source column.
2. The EHD source chamber is isolated from the cleaning chamber by differential pumping.
3. Exposure of the cleaning chamber to vapor or sources of contamination originating in the EHD source chamber are eliminated or minimized.
4. A surface is exposed to the EHD beam-line only at small regions where particles exists. Unnecessary exposure of target areas that do not contain particles is avoided.

An in-situ metrology inspection system can be installed in the cleaning chamber that verifies removal of nanometer-sized particles. A laser-based system can be used for this purpose that detects the presence or absence of a particle after exposure to the microdroplet beam. Electrostatic or other means to collection removed particles must be implemented to insure that the particle has not moved to another location on the mask.

The EHD microdroplet beam mounted in the EHD column chamber 10 is prefocused by the source lens 15 and further collimated by the beam column lens 25, subsequently passing through an aperture in the orifice plate 30. A beam shutter 20, in conduction with a rectangular slit valve, is used to isolate the beam from the cleaning chamber 50 when cleaning is not desired. The beam line 35 which enters the cleaning chamber 50 passes through the electrostatic collector 40 and impacts the substrate directly beneath the collector. A set of electrostatic deflection plates 60 is used to deflect, wiggle or raster the beam line 35 at the target. An x-y positioning stage 45 is used to move the substrate containing residue particles beneath an aperture located in the collector mask 40. Wafers, photomasks or other surfaces to be cleaned or placed in or removed from the cleaning chamber 50 through a rectangular slit valve using a vacuum robot.

### Linear Slit Emitter

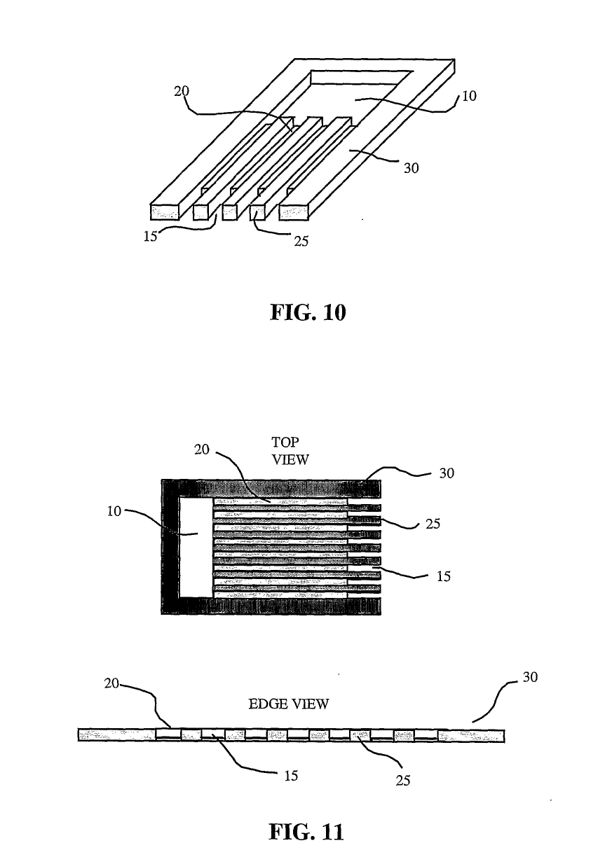

An alternative to a single capillary nozzle or a linear array of discrete nozzles is an emitter design based on a linear slit geometry. This invention involves the fabrication of an integral linear slit device that can replicate the microdroplet emission from tens or hundreds of nozzles fabricated individually. Several techniques for linear slit fabrication are available including but not limited to photochemical etching (PCE) and microelectromechanical (MEMs) machining methods. One embodiment of a linear slit design is shown in FIG. 10 referred to as a slit rake 30. The rake thickness should be kept as small as possible, 0.003 mil or less to minimize the voltage applied to the rake fingers 25 necessary to achieve the electric field required to emit microdroplets in the desired size range.

Solution is introduced into the rake plenum 10 and flows through the grooves 20 filling the gaps 15. When the solution wets the tips of the fingers 25, the high electric field causes the solution to atomize producing multiple beams of charged microdroplets. FIG. 11 shows a top and edge view of the slit rake 30. The slit rake can be fabricated by PCE methods from stainless steel or other suitable material. If machined by MEMs technology, a preferred material for the rake would be silicon or paralyne. The overall length of the rake is determined by the number of emitting fingers required to cover the desired processing area. A preferred groove 20 depth is 0.002 inches or less. The gap 15 length and finger 25 length should be about 0.004 inches or smaller. The slit rake is bonded between an upper and lower plate (not shown) to prevent solution from leaking at the edges. The rake design is well-suited for atmospheric operation because the high flow of solution from multiple emission sites would overburden a vacuum system.

### Method for Improving EHD Nozzle Emission Stability and Reducing Contamination Buildup at Tips

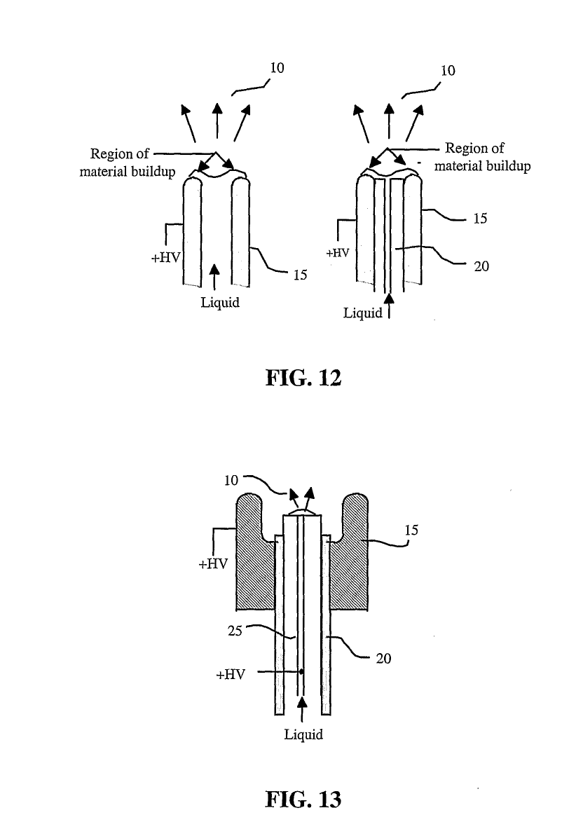

This invention relates to significant improvements in the overall performance (stability) of EHD microdroplet nozzle and slit emitters. Earlier designs suffered from the persistent buildup of deposits at the emitter tip requiring frequent cleaning to sustain consistent and repeatable performance. With the aid of FIG. 12, a design is described that eliminates the formation of deposits observed with the earlier emitters. In the earlier design shown, a beam or spray 10 occurs when the solution makes electrical contact with the metal capillary 15. During the process of charge transfer at the tip, electrochemically activated deposits form at the tips and inside of the metal capillary rim or where a fused silica capillary 20 contacts the metal nozzle 15.

Fluctuations in emission levels, attributed to non-uniform spreading of the conductive solution over the fused silica capillary 20 surface, was another problem encountered with earlier emitter designs. Stable emission currents require that repeatable wetting tale place at the charge transfer interface. Good wetting is not always achieved as manifested by instabilities in the DC current levels.

A design which prevents materials deposits at nozzles tips and eliminates wetting problems is shown in FIG. 13. The improvement arise, in part, by allowing the fused silica capillary 25 to protrude slightly above the inner metal capillary support 20. In this design, the solution is charged by applying high voltage to a chemically inert electrode placed directly in the solution. When the field between the charged surface and the extractor electrode is high enough, a charged microcluster beam leaves the bore of the fused silica capillary. To sustain a continuous spray, charge (electron) transfer occurs at the remote electrode and not at the emitter tip as in previous designs. Using the emitter design shown in FIG. 13, the spraying mechanism does not require any electrochemical reactions, involving charge transfer, from taking place at the emitter tip. Unlike the previous emitter designs, the conductive path for stable flow of current no longer depends on the unpredictable wetting conditions at the emitter tip. A wire electrode, placed in the solution reservoir, should function solely as a sink for electrons and play no adverse chemical role in the electrode reaction. Preferred materials for the electrode are gold or platinum.

Besides greatly improving the incidence of debris buildup at the tips and improving emission stability, the new design has other unexpected benefits including:
a. Removal of wetting problems causing fluctuating beam currents.
b. Better reproducibility in emitter-to-emitter performance by removing the dependency on metal capillary-solution wetting conditions.
c. Metal capillary manufacturing tolerances depend less on rim uniformity and thickness, concentricity etc. for consistent emitter performance.
d. Electrochemical corrosion of the inner metal capillary is eliminated allowing the capillary to be manufactured from inexpensive materials such as stainless steel or aluminum rather than platinum or platinum alloys.
e. Microdroplets are no longer subjected to metallic impurities formed when metal capillaries react with solutions during charge transfer. This has special relevance for semiconductor wafer cleaning.

For better control, the improved design requires application of high voltage to, not only the solution, but also to the emitter cap 15 enclosing the fused silica 25 and inner metallic capillary 20. The electric field formed at the outer metal cap 15 reduces microdroplet beam spreading. Additionally, the fused silica emitter 25 is shielded from backstreaming electron impacts by the attractive field of the surrounding emitter cap 15.

### Dual Chamber Configuration for Improving EHD Cleaning Performance

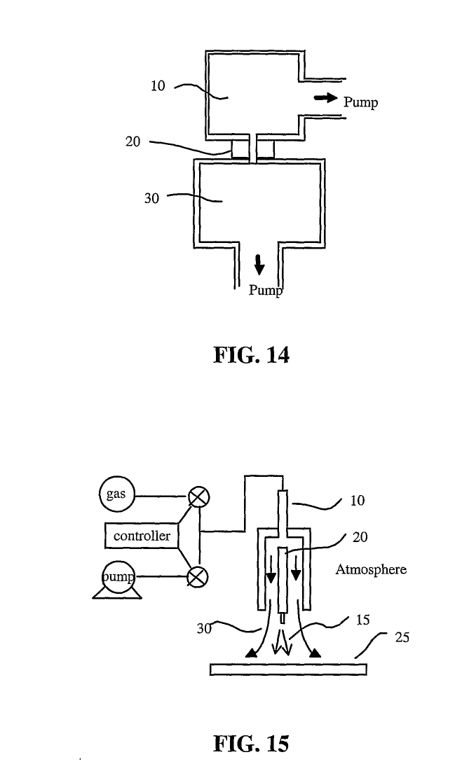

FIG. 14 illustrates a method for isolating an EHD emitter apparatus form a work-piece or target substrate that undergoes surface modification or cleaning using a charged microdroplet beam.

In FIG. 14, the upper chamber 10 houses the EHD emitter head apparatus and the lower chamber 30 encloses a workpiece or target substrate. The two chambers are joined by a transition block 20 that contains a single aperture, multiple apertures or a narrow slit which allows a beam or beamlets to pass from the upper chamber to the lower chamber. The chambers 10 and 30 have separate evacuation ports for differential pumping that can individually control the pressure in each chamber. Benefits from this configuration include elimination of contaminants originating in the upper chamber 10 and microdroplet source from entering the substrate chamber 30.

### Atmospheric Operation of EHD Emitters

At sufficiently low voltage, nozzle or slit emitters can be operated at atmospheric conditions for cleaning or modifying a target or workpiece. FIG. 15 shows a configuration for atmospheric operation of an EHD source. The slit or nozzle assembly 20 is positioned above but in close proximity to the workpiece 25. When desirable, a concentric gas flow 30 provided by a coaxial flow chamber 10 can be directed onto the workpiece. The gas flow can be used to provide additional energy to the microdroplet beam 15 thereby increasing the microdroplet velocity. In addition, the gas flow 30 can purge air in the region surrounding the emitter 20 replacing it with a gas exhibiting high electrical breakdown resistance. Used in the latter function, higher voltages can be applied to the emitters before the onset of electrical discharge occurs in the emitter region. Also, gas flow can aid in the resuspension and collection of impacted particles when the EHD source operates in the surface cleaning mode using an electrostatic particle trapping plate.

Gas flow to the nozzle region is controlled by means of electrically operated valves connected to a source of gas and a vacuum pump. The atmospheric source depicted in FIG. 15 can be easily translated in x-y directions for location above desired workpiece regions. Further, the source can be tilted so that the microdroplet beam impacts the workpiece at desired angles of incidence.

Electrostatic Collection of Charged Particles Dislodged from Surface Impacted by Microdroplet Beam

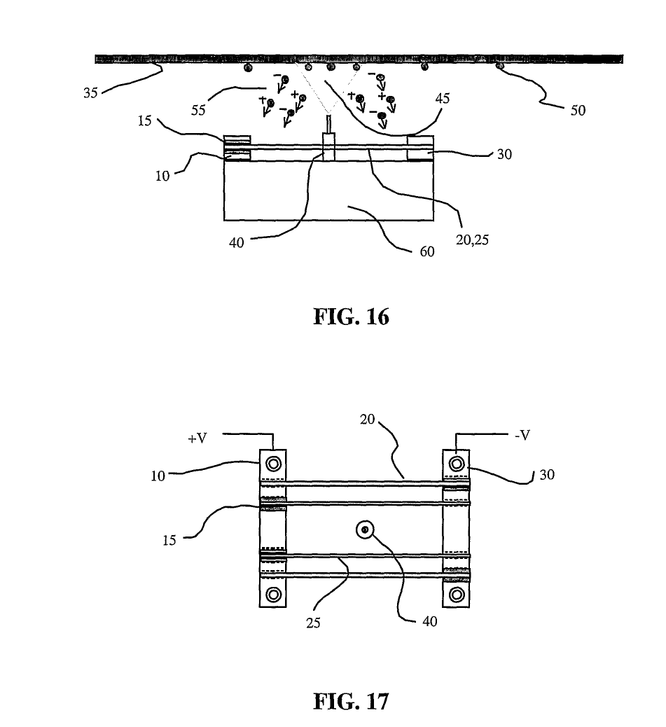

FIG. 16 is a side view of the basic concept and apparatus for collecting particles dislodged from a surface impacted by a charged microdroplet beam. In a preferred embodiment of the present invention, particles or debris 55 removed from a surface 35 impacted by a microdroplet beam 45 are electrostatically attracted to charged, conducting rods 20, 25 of opposite polarity.

The electrostatic collection assembly attached to the emitter housing 60 consists of a plurality of conducting, metallic elements (rod, wire, strips) 20, 25 connected to power sources capable of applying positive and negative potentials to respective elements. In one embodiment of the invention, the conductive elements may be coated with a dielectric film such as paralyne to prevent re-deposition of particles on the surface by electrostatic repulsion effects. Initially uncharged particles and debris 55 removed from the surface 35 after impact by a charged microdroplet beam 45 can carry a net positive or negative charge. Particle charging can occur by charge transferred from the primary microdroplet beam 45, from secondary electrons generated at the surface 35, from electrons emitted by a neutralization source (thermionic emitter or low energy electron flood source) or from bipolar ions present in the impact region arising from air ionizers. Charged debris 55 is attracted to the electrostatic elements 20, 25 by the electric field established between the respective elements.

FIG. 17 shows a top view of the electrostatic collection assembly which mounts to the EHD emitter housing 60. The assembly consists of two conducting rails 10 and 30 connected to power sources of opposite polarity (+,−). The electrostatic collection rods 20 and 25 are joined to the conducting rails in one or more pairs. Rods with positive applied potential 20 are insulated from the conducting rail held at a negative potential 30 by means of an insulating sleeve 15 made from a dielectric material such as Teflon, ceramic or plastic. Rods with negative applied potential 25 are insulated from the conducting rail held at a positive potential 10 by means of an insulating sleeve 15. The EHD emitter 40 is positioned between the pairs of collection rods in such a manner that the electrostatic fields of the rods do not interfere with the electric field at the emitter 40. Dislodged debris 55 carrying a net negative charge is attracted to the positively charged collection rods 20 and debris carrying a net positive charge is attracted to the negatively charged collection rod 25. This electrostatic collection arrangement can be installed on EHD emission sources which operate in an air, gas or vacuum environment. It should be pointed out that the electrostatic collection system described above, displayed in FIGS. 16 and 17, can be extended to accommodate the collection of debris dislodged from surfaces using a geometrical array of multiple EHD microdroplet sources disposed in a linear or rectangular arrangement.

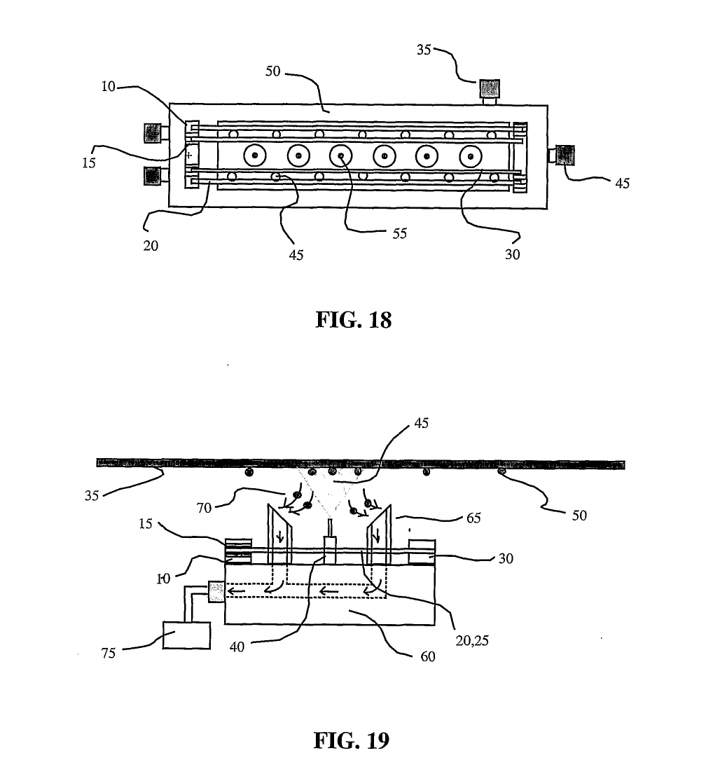

One embodiment of a geometrical array of multiple EHD emitters is shown in FIG. 18 showing a linear array of six EHD emitters although the number of emitters could be less or extended to more than six. The array 50 shows six EHD nozzles 55 spaced equally apart although the distance between nozzles could be varied depending on the surface cleaning application. In the diagram 14 vacuum updraft openings 45 are depicted.

### Vacuum Intake Debris Collection

A second embodiment of the debris collection system described in the previous section, applicable to atmospheric surface preparation applications, is shown schematically in FIG. 19. In this configuration, a plurality of vacuum conduits 65 are positioned atop the EHD emitter source 40 with intake openings facing the target substrate 35. Connecting the vacuum conduits to a vacuum pump 75 creates an updraft, pulling air and entrained debris 70 into the conduit intake openings. In conjunction with the electrostatic collection assembly, the vacuum updraft conduits 65 assist in collection of debris 70. The number and disposition of vacuum conduits 65 can be extended to accommodate the collection of debris dislodged from surfaces using a geometrical array of multiple EHD microdroplet sources.

### EHD Emitter Structure for Anchoring Emission Sites

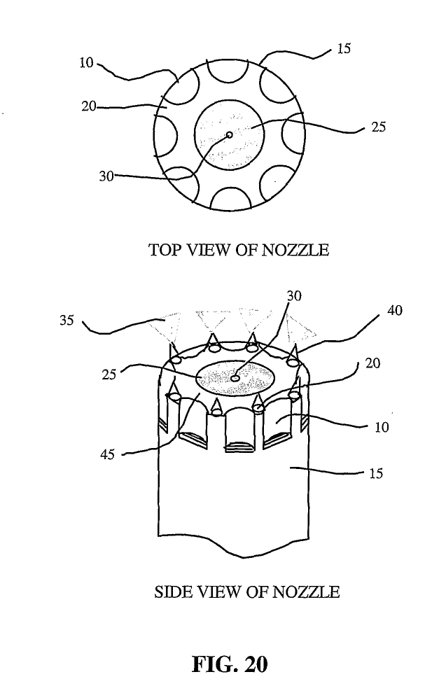

A preferred mode of EHD microdroplet emission for surface preparation is a so-called “crown” emission. In this mode, multiple emission sites are located at the periphery (rim) of the EHD emission nozzle where the electric field has its highest value. The number of emission sites scale with the high voltage applied. Although the multiple emission site mode can remain stable for long periods, the number of sites can change or appear to rotate under the influence of a varying field or changes in the wetting characteristics at the emitter rim boundary. For stability, it is desirable to anchor or fix the number of sites for better emission control. A preferred method for accomplishing “crown” emission stability is to modify the emitter tip region by micromachining “fixed” areas of the emitter rim that enhance the electric field at precise locations which are less susceptible to changes induced by fluid movement or small changes in the physical dimensions of the emitter tip. A method to precisely anchor the emission sites to specific locations at the EHD emitter tip is disclosed in FIG. 20.

A top view of a PEEKsil EHD microdroplet emitter 15 is shown in FIG. 20 displaying 8 emission plateaus 20 arranged in a symmetrical pattern around the periphery of the EHD nozzle. The plateaus 20 are created by micromachining (using a laser or microtools) grooves 10 along the nozzle shaft 15 and parallel to the nozzle axis. Solution exiting under pressure from the orifice 30 in the bonded fused silica tubing 25 flows across the wetted surface 45 onto the plateaus 20 exposed to a high electric field. The machined voids (spaces) between the wetted plateaus remain unfilled due to the hydrophobic nature of the PEEK outer tubing 15. Emission sites for individual microdroplet beamlets 35 are therefore effectively anchored only to the wetted plateau regions 20 where conditions favor formation of liquid conical protrusions 40. Although 8 plateaus are shown in FIG. 19 corresponding to eight emission sites, the preferred number of machined plateaus 20 (emission sites) lie in the range from 4 to 12.

### Atmospheric/Vacuum EHD Microdroplet Source Assembly

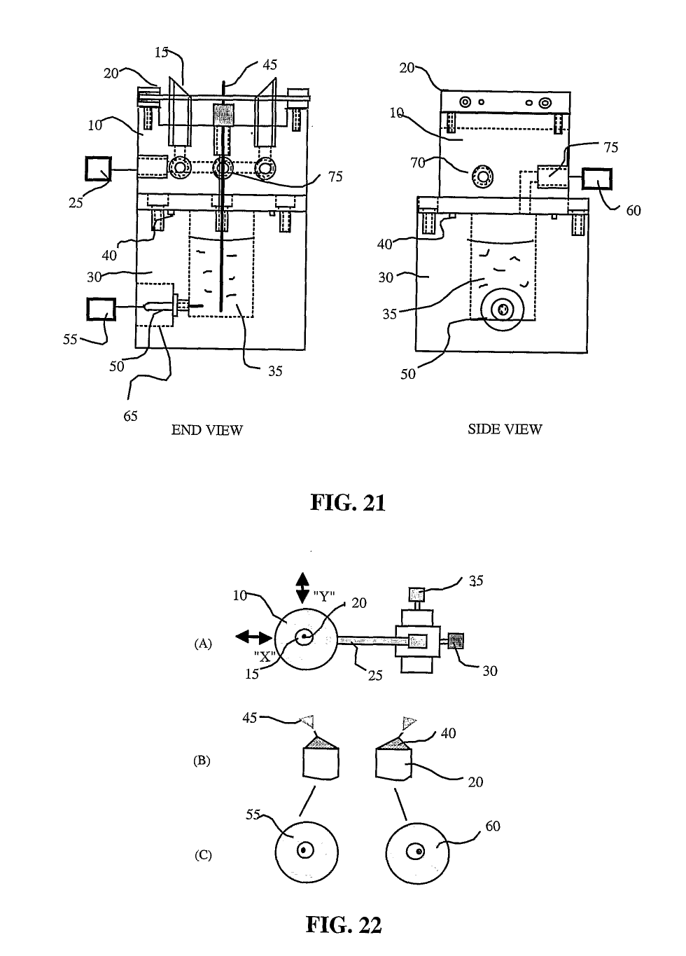

FIG. 21 is a diagram showing the basic apparatus for generating charged microdroplets used in surface preparation applications e.g., cleaning, texturing, deposition, surface drying and surface chemistry modification. In the preferred embodiment, the apparatus consists of two main modules, the reservoir module 30 and the EHD emitter or source module 10. The reservoir module consists of a chamber 35 containing the fluid supply for dispersion by the emitter module, a means for applying voltage to an electrode 65 immersed in the solution and a pressure port 75 for applying vacuum or positive pressure to the reservoir solution. The electrode 65 is preferably an inert metal, e.g. gold or platinum, that prevents chemical interaction between the electrode 65 and fluid supply 35. The electrode 65 is connected to a hermetic connector 50. A power source 55 is used to apply voltage to the hermetic connector 50. The reservoir module 30 is sealed to the EHD emitter module 10 by means of an o-ring type seal 40.

The EHD emitter module 10 consists of a PEEKsil emitter assembly 45, vacuum updraft conduits 15 and an electrostatic collector assembly 20. A vacuum source 25 is connected to the vacuum port 70 to provide a means for intaking debris dislodged from a surface impacted by the microdroplet beam. A pressure source 60 is connected to the pressure port 75 as a means for pressurizing the fluid supply 35 in the reservoir chamber.

The high field at the nozzle tip is achieved by applying high voltage to the connector 50. The pressure applied through the port 75 to the reservoir chamber is controlled by two valves connected to a source of pressure and vacuum. A pressure sensor at the input of the pressure port is set by a computer controlled program.

Depending on the desired emission mode, single cone-jet or crown emission, the charged droplet generating apparatus is preferably operated with voltages ranging from 3 to 8 kV with emission currents ranging from about 0.05 to over 3 μA using a single EHD emitter.

### Microdroplet Beam Steering

As an alternative to electrostatic beam steering of Taylor cone-jet sprays, the present invention employs a means for mechanical steering of the beam as shown in FIG. 22. Mechanical beam steering is accomplished by modifying the electric field at the Taylor cone by changing the concentric centering of an EHD nozzle emitter 20 within a circular aperture 15 machined into an extractor electrode 10. The electrostatic symmetry of the nozzle-concentric aperture 15 is converted to an asymmetrical arrangement by adjusting the extractor electrode position to offset the nozzle tip 20 from axial symmetry.

The extractor electrode 10 is coupled by means of linkage 25 to a miniature motorized translation stage. The motion of the translation stage is controlled by an “X” motor 30 and a “Y” motor 35. FIG. 22b and c show how the Taylor cone 40 jet spray is diverted off-axis 45, 40 when the extractor electrode 10 has reached a final “+X” position 55 or a final “−X” position 60. By use of mechanical beam steering, the microdroplet beam can be directed to specific target areas on a substrate material.

### Process Chemistries

The preferred chemistries for microdroplet formation include, but are not limited to, solutions which consist of one or more of the solvents listed in Table 3. In addition to formulations which involve the pure solvent or mixing one or more of the solvents in varying proportions, solutes can be added to the solution chemistry as dissolved electrolytes in order to vary the conductivity of the overall process chemistry. Examples of chemicals which can be used to vary solution conductivity are listed in Table 4 Solution conductivities can range from 0.05 to 10⁵ μS/cm. Unlike atmospheric operation, solutions with a low vapor pressure are preferred for vacuum operation of EHD sources in background pressures of 10⁻⁴ to 10⁻⁵ torr.

TABLE 3
Process Solution Chemistry Properties.

| | | | BP | FP | Dens. | ST | | Visc. |
|---|---|---|---|---|---|---|---|---|
| Chemical | Formula | MW | (° C.) | (° C.) | (g/cc) | (dyne/cm) | ε | (cp) |
| N,N-dimethylacetamide | C₄H₉NO | 87.1 | 165 | −20 | 0.938 | 33.5 | 37.8 | 0.93 |
| Propylene Carbonate | C₄H₇O₃ | 102.1 | 242 | −49 | 1.2 | 40.9 | 64 | 2.5 |
| N-Methyl-2-Pyrrolidone | C₅H₉NO | 99 | 202 | −24.4 | 1.028 | 41 | 32 | 1.65 |
| N-Butylamine | C₄H₁₁N | 73 | 77.7 | −50.5 | 0.74 | 23.9 | 5.4 | 0.59 |
| Hydrogen Peroxide | H₂O₂ | 34 | 226 | −27 | 1.132 | 74.5 | 121 | 1.11 |
| (35%) | | | | | | | | |
| Water | H₂O | 18 | 100 | 0 | 1.0 | 73 | 80 | 1.0 |
| Isopropyl Alcohol (IPA) | C₃H₈O | 60 | 83 | −88 | 0.785 | 22 | 20 | 2.4 |
| Methanol | CH₄O | 32 | 65 | −98 | 0.793 | 22 | 33 | 0.6 |
| Ethylene Glycol | C₂H₆O₂ | 62 | 197 | −12 | 1.115 | 48 | 38 | 21 |
| Formamide | CH₃NO | 45 | 210 | +2.5 | 1.133 | 58 | 84 | 3.76 |
| Hydroyxlamine | | | | | | | | |

ε = Dielectric constant, MW = molecular weight, BP = Boiling point, FP = Freezing point, ST = surface tension
Visc. = viscosity and Dens. = density.

TABLE 4
Process Solution Additives

Hydrochloric Acid
Nitric Acid
Hydrofluoric Acid
Ammonium Hydroxide
Ammonium Fluoride
Acetic Acid

### One Process Chamber Embodiment

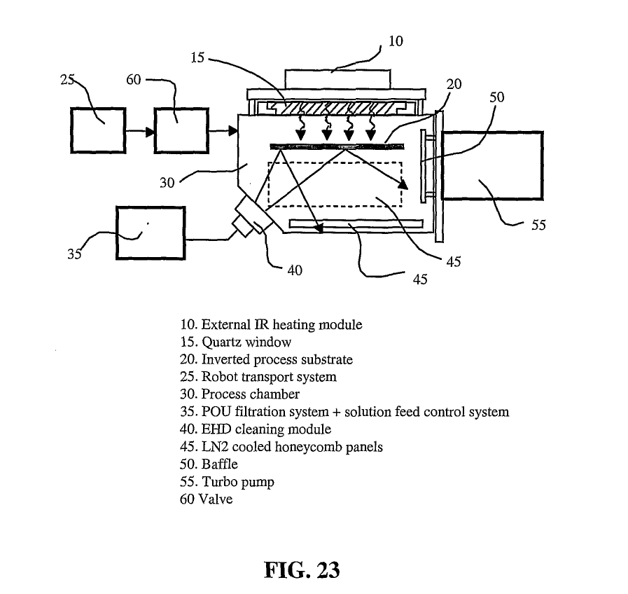

### (See FIG. 23)

### Another Process Chamber Embodiment

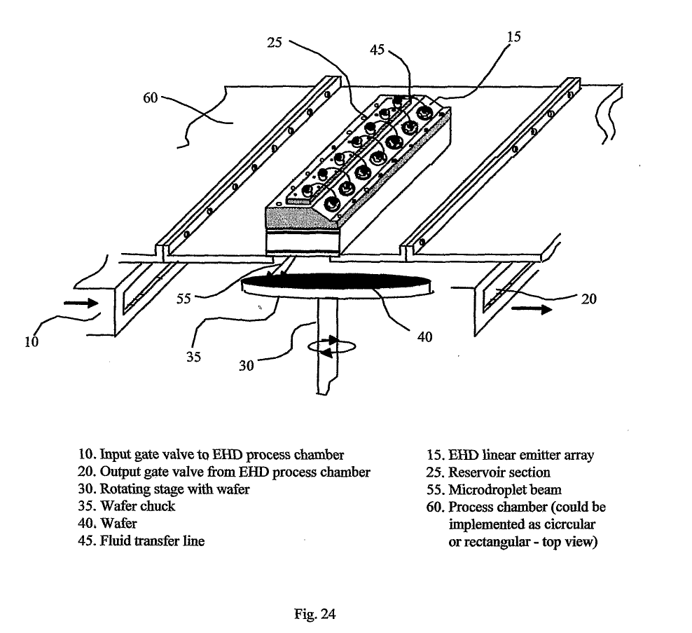

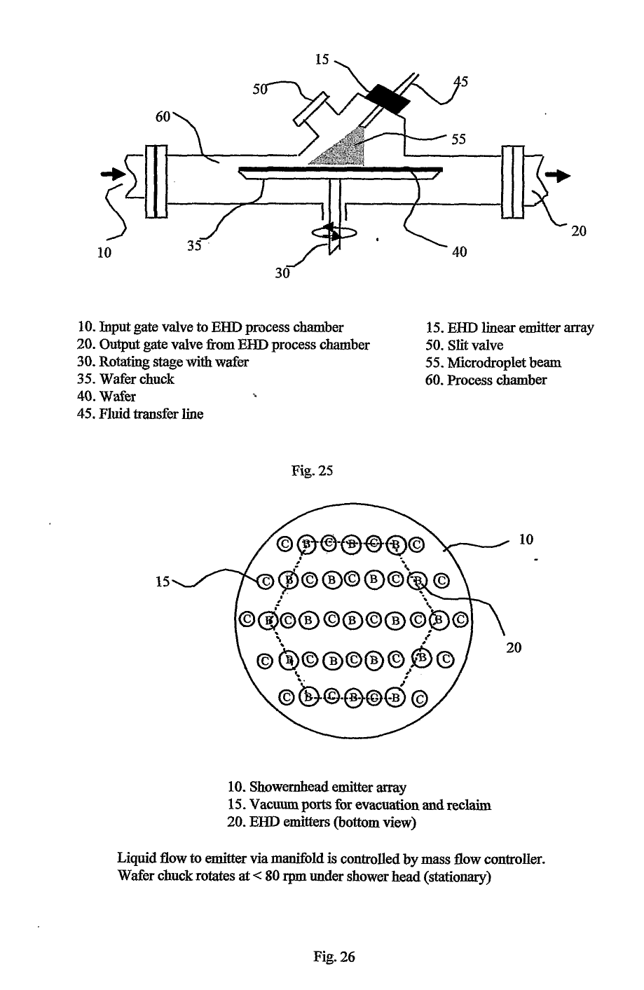

FIG. 24 shows a second process chamber embodiment. In this design, a linear array of EHD emitters covers a portion of a rotating workpiece holder. This low-profile chamber can be operated in atmospheric, vacuum, or gas environments. FIG. 25 is a cross section view of this chamber.

### Showerhead Nozzle Array

FIG. 26 is a view of an integrated EHD emitter and vacuum updraft collection array that provides full coverage above a rotating workpiece such as a semiconductor wafer.

### Surface Tension Gradient (Marangoni) Drying Improvement

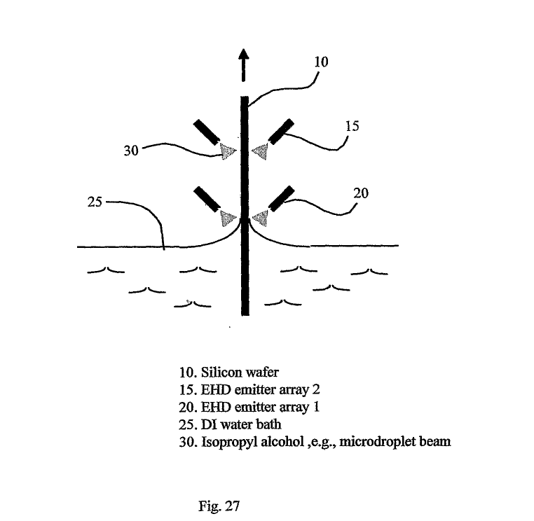

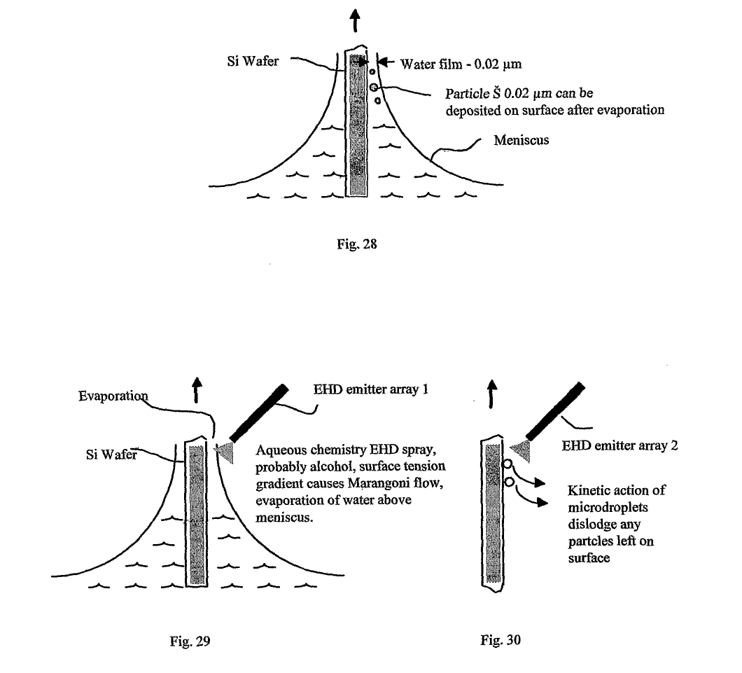

FIG. 27 through FIG. 30 show EHD emitters assisting a Marangoni drying process. The addition of microcluster beams as a final “sweep” of the thin film of liquid adds a kinetic element to ensure that particles trapped in the final liquid film do not deposit onto the substrate surface and cause “watermarks”.

What is claimed is:

1. A system to remove contaminants from a surface, the system comprising:
a source to generate a beam of clusters to said surface, said source having an opening;
a feed system to feed a liquid to said opening; and
a device to generate an electric field to exert, upon liquid fed to a vicinity of said opening, electrostatic forces higher than a surface tension of said liquid, and a vacuum chamber that houses the source and the surface.

2. A method for removing contaminants from a surface, the method comprising: feeding a liquid to a low pressure location where a beam of clusters is generated; generating said beam of clusters by exerting, upon said liquid fed to said location, electrostatic forces higher than a surface tension of said liquid; and directing said beam of clusters to said surface.
